(19) United States
(12) Patent Application Publication
(10) Pub. No.: US 2010/0168509 A1
(43) Pub. Date: Jul. 1, 2010

(54) TREATMENT OF KINK RESISTANT TUBING

(76) Inventor: Charles C. Kuyava, Eden Prairie, MN (US)

Correspondence Address: AMS RESEARCH CORPORATION, 10700 BREN ROAD WEST, MINNETONKA, MN, 55343, US

(21) Appl. No.: 12/646,116

(22) Filed: Dec. 23, 2009

Related U.S. Application Data
(60) Provisional application No. 61/140,367, filed on Dec. 23, 2008.

(51) Int. Cl. A61F 5/00

(52) U.S. Cl. 600/40

(57) ABSTRACT

An implantable penile prosthesis having a protective surface protecting an inflatable cylinder from damage caused by repeated contact with kink resistant tubing. The protective surface is fabricated of a low friction material to reduce abrasive damage caused by the kink resistant tubing repeatedly rubbing against the inflatable cylinder. The protective surface can have cushioning or force distributing qualities to reduce impact forces caused by the kink resistant tubing repeatedly impacting the inflatable cylinder. The protective surface can be adapted to expand and contract with the inflation or deflation of the inflatable cylinder such that the protective surface does not deform the inflatable cylinder nor does the protective surface separate from the inflatable cylinder as the inflatable cylinder changes in size.

### PRIORITY CLAIM

The present application claims priority to U.S. Provisional Application Ser. No. 61/140,367, filed Dec. 23, 2008, and entitled “TREATMENT OF KINK RESISTANT TUBING (KRT),” which is herein incorporated by reference in its entirety.

### FIELD OF THE INVENTION

The present application is directed to an implantable penile prosthesis having kink resistant tubing. More specifically, the present invention is directed to a protective surface preventing the kink resistant tubing from damaging the implantable penile prosthesis while also maintaining the effectiveness of an anti-bacterial coating applied to the implantable penile prosthesis.

### BACKGROUND OF THE INVENTION

Implantation of an implantable penile prosthesis (IPP) is a common surgical procedure for treating erectile dysfunction and other penile ailments. Typically, implantable penile prostheses and comprise an inflatable cylinder connected to a pump by kink resistant tubing. In other versions, an implantable penile prosthesis can further comprise a reservoir that stores solution for inflating and deflating the inflatable cylinder with a pump that is connected to the inflatable cylinder via kink resistant tubing. These versions of the implantable penile prosthesis are available under the trade names AMBICO and AMS 7000 from American Medical Systems of Minnetonka, Minn. Typically, the pump is not affixed to any fixed bodily structure and freely hangs from the inflatable cylinder and the reservoir by the kink resistant tube. Movement of the unfixed pump or movements of the patient can cause the kink resistant tubing to repeatedly impact or rub against the inflatable cylinder, ultimately resulting in damage to the inflatable cylinder. Similarly, damage can occur within the kink resistant tubing connecting the reservoir to the pump.

The kink resistant tubing can comprise a sleeve preventing the kink resistant tubing from directly contacting the inflatable cylinder. The sleeve generally comprises materials with low frictional coefficients such as, for example, expanded PolyTetraFluoroEthylene (ePTFE), to minimize the abrasive forces applied to the inflatable cylinder as the kink resistant tubing rubs against the inflatable cylinder. The sleeve typically does not cover the entire length of the kink resistant tubing, but instead, covers only a predetermined portion of the kink resistant tubing that is most likely to rub against the inflatable cylinder.

One drawback of the sleeve approach is that tissue in-growth into the sleeve can occur if any portion of the sleeve extends beyond the corporotomy in the corpora cavernosa. Tissue in-growth complicates subsequent revision surgeries as the in grown tissue and the portion of the sleeve containing the in grown tissue must be first excised by the surgeon before the revision surgery can proceed, lengthening the procedure and increasing the risk of complications. Surgeons often seek to avoid tissue in-growth by removing any portion of the sleeve extending beyond the corporotomy to avoid tissue in-growth. However, this additional removal step lengthens the time required for the initial procedure and can damage the kink resistant tubing.

An additional drawback is that materials having low frictional coefficients as used in the sleeve can affect the application of an antibiotic coating to the implantable penile prosthesis. Antibiotic coatings such as, for example, the INHIBIZONE antibiotic surface treatment produced by American Medical Systems of Minnetonka, Minn. can be absorbed more readily by some materials than others. For example, ePTFE far more readily absorbs INHIBIZONE than the other materials used in the implantable penile prosthesis often resulting in an uneven distribution of the antibiotic coating. Uneven distributions of the antibiotic coating can compromise the effectiveness of the overall antibiotic coating.

Consequently, there is a need for an apparatus capable of protecting the inflatable cylinder from damage due to repeated contact with the kink resistant tubing. There also exists a need for an apparatus that avoids tissue in-growth and maintains or improves the effectiveness of the antibiotic coating on the implantable penile prosthesis.

### SUMMARY OF THE INVENTION

One representative embodiment of the present disclosure is directed to an implantable penile prosthesis having a protective surface protecting an inflatable cylinder from damage caused by repeated contact with kink resistant tubing. The protective surface generally comprises materials having low frictional coefficients to reduce abrasive damage caused by repeated rubbing of the kink resistant tubing against the inflatable cylinder. The protective surface can also comprise materials having cushioning or force distributing qualities to reduce impact forces caused by repeated impact of the kink resistant tubing against the inflatable cylinder. The protective surface can be adapted to expand and contract with the inflatable cylinder such that the protective surface does not deform the inflatable cylinder nor does the protective surface separate from the inflatable cylinder as the inflatable cylinder changes in size.

In another representative embodiment, an implantable penile prosthesis can comprise an inflatable cylinder, a pump, and kink resistant tubing connecting the inflatable cylinder to the pump. The inflatable cylinder can further comprise a protective surface disposed on an outer surface of the inflatable cylinder proximate the junction of the inflatable cylinder and the kink resistant tubing. The protective surface can cover the entire exterior of the inflatable cylinder or alternatively, can cover only a predetermined area to protect a portion of the inflatable cylinder exterior most likely to contact the kink resistant tubing. The protective surface can also be sized so as to minimize the effect of the materials used in the protective surface on the effectiveness of an antibiotic coating applied to the implantable penile prosthesis. The protective surface in an unstretched state can be affixed to the inflatable cylinder such that the protective surface can expand when the inflating inflatable cylinder is inflated. Alternatively, the protective surface in a stretched state can be affixed to the inflatable cylinder when inflated such that the protective surface can compress when the inflatable cylinder is deflated.

In another representative embodiment, an implantable penile prosthesis can further comprise a reservoir, an inflatable cylinder and a pump operably connected with kink resistant tubing. The inflatable cylinder can further comprise a second protective surface on the inflatable cylinder at a predetermined location in which the kink resistant tubing joining the pump to the reservoir contacts the inflatable cylinder. Alternatively, a single protective surface can cover areas where the inflatable cylinder is most likely to contact the kink resistant tubing leading to the pump and the kink resistant tubing extending between the reservoir and pump.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE DRAWINGS

The invention can be completely understood in consideration of the following detailed description of various embodiments of the invention in connection with the accompanying drawings, in which:

FIG. 1 a perspective view of a prior art implantable penile prosthesis having a sleeve for protecting an inflatable cylinder.

FIG. 2 a perspective view of a prior art implantable penile prosthesis having a sleeve for protecting an inflatable cylinder.

FIG. 3 a perspective view of a representative embodiment of an implantable penile prosthesis having a protective surface for protecting an inflatable cylinder according to the present invention.

FIG. 4 a perspective view of a representative embodiment of an implantable penile prosthesis having a protective surface for protecting an inflatable cylinder according to the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

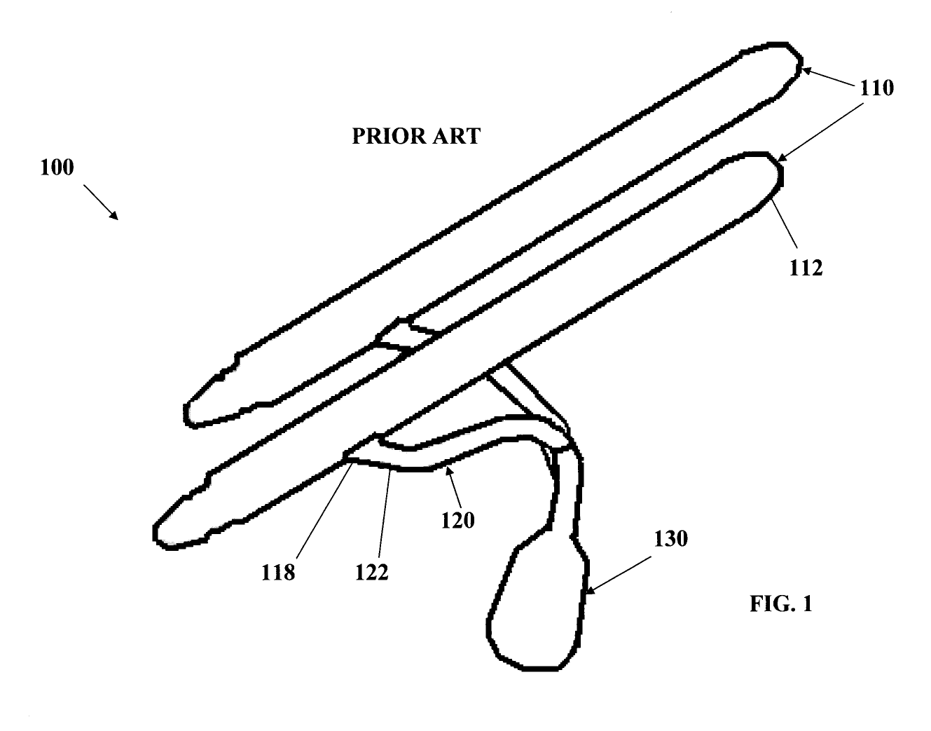

As illustrated in FIG. 1, a conventional implantable penile prosthesis 100 of the prior art comprises a pair of inflatable cylinders 110 and a pump 130. The implantable penile prosthesis 100 further comprises kink resistant tubing 120 linking the inflatable cylinders 110 to the pump 130. The kink resistant tubing 120 comprises a sleeve 122 extending from a junction 118 of the kink resistant tubing 120 and the inflatable cylinder 110 to a predetermined length along the kink resistant tubing 120. The length of the kink resistant tubing 120 covered by the sleeve 122 is determined based on the portion of the kink resistant tubing 120 most likely to contact the inflatable cylinder 110 during a patient's ordinary movement.

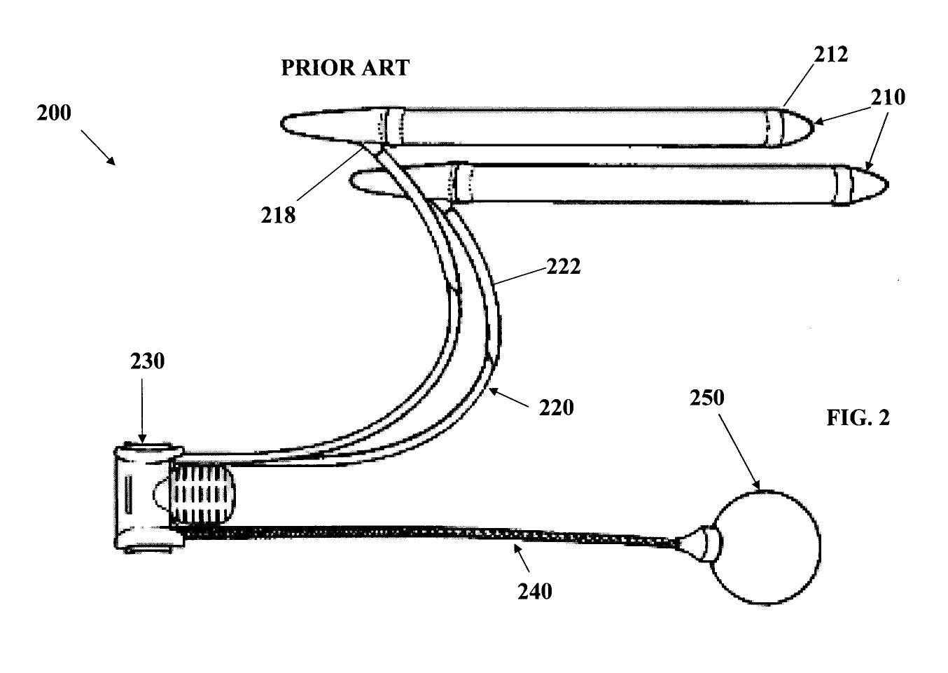

As depicted by FIG. 2, another embodiment of a conventional implantable penile prosthesis 200 further comprises a reservoir 250 linked to a pump 230 by kink resistant tubing 240. Pump 230 is operationally linked to each inflatable cylinder 210 by kink resistant tubing 220. Kink resistant tubing 220 is at least partially covered by sleeves 222 extending from a junction 218 of the kink resistant tubing 220 and each inflatable cylinder 210 to a predetermined length.

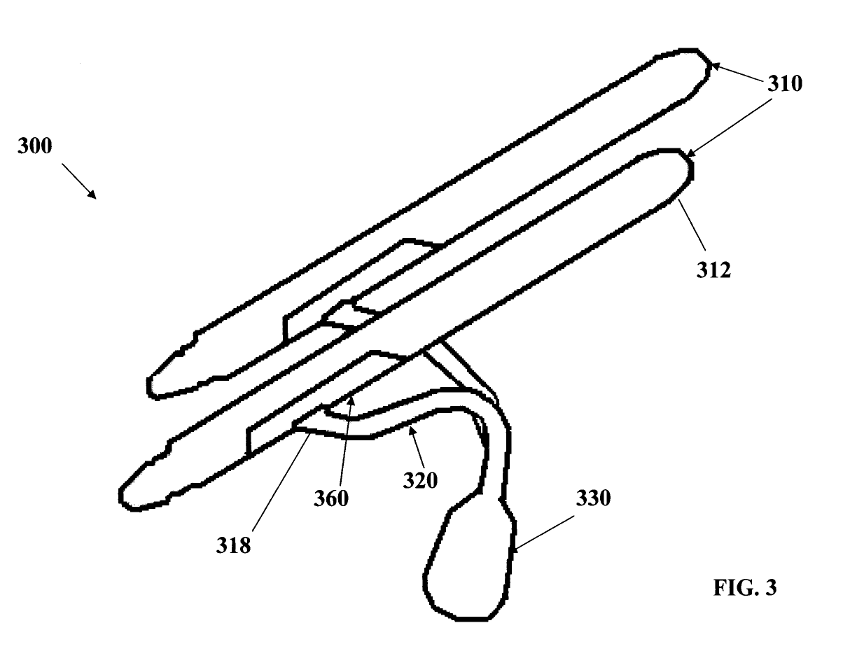

As depicted by FIG. 3, a representative embodiment of an implantable penile prosthesis 300 according to the present disclosure comprises an inflatable cylinder 310 and a pump 330. The implantable penile prosthesis 300 further comprises kink resistant tubing 320 linking the pump 330 to each inflatable cylinder 310. Inflatable cylinders 310 each include a protective surface 360 on an exterior portion 312 of the inflatable cylinder 310. Protective surface 360 can be affixed to the inflatable cylinders 310 by adhesive bonding or any other suitable means of permanently affixing the protective surface 360 to the exterior 312 of the inflatable cylinders 310.

Protective surface 360 generally covers a predetermined area on the exterior portion 312 of the inflatable cylinder 310 surrounding a junction 318 of the kink resistant tubing 320 and the inflatable cylinder 310. The predetermined area is determined based on the area of the inflatable cylinder 310 most likely to contact the kink resistant tubing 320. Protective surface 360 can have width ranging from a 1 cm width intended to encompass 95% of the potential area on the inflatable cylinder 310 typically contacted by kink resistant tubing 320 all the way to width of 2 cm that encompasses 100% of the area on inflatable cylinder 310 typically contacted by the kink resistant tubing 320. Alternatively, protective surface 360 can be expanded to cover the entire exterior portion 312 of the inflatable cylinder 310.

Protective surface 360 can comprise ePTFE, poly(p-xylylene) (PARYLENE), or other suitable materials having low frictional coefficients. A representative embodiment of protective surface 360 comprises a 2 cm wide PARYLENE surface providing a tough and easily adhered protective surface 360 having a low frictional coefficient. PARYLENE is frequently used in the inflatable cylinders 310 of conventional implantable penile prosthesis for other purposes, thereby reducing the manufacturing conversion costs of adding a PARYLENE protective surface 360 to the exterior portion 312 of each inflatable cylinder 310. Another representative embodiment can comprise a 2 cm wide ePTFE protective surface 360 providing the same protection as the ePTFE sleeve 122 on conventional implantable penile prosthesis 100.

Implantable penile prosthesis 300 as a whole can be treated with an antibiotic coating such as INHIBIZONE. The width of protective surface 360 is adjusted depending on the zone of inhibition created by the antibiotic coating and the absorption of the antibiotic coating by the material comprising the protective surface 360. Protective surface 360 comprising material that does not readily uptake the antibiotic coating can be limited in width to the size of the zone of inhibition created by that antibiotic coating. Protective surface 360 can also be limited in width if the protective surface 360 comprises a material having a better uptake of the antibiotic coating than the other materials used in the implantable penile prosthesis 300 to maintain a balanced distribution of the antibiotic coating.

Protective surface 360 is sufficiently elastic to expand and contract with the inflatable cylinder 310 as the inflatable cylinder 310 is inflated or deflated. The protective surface 360 in an unstretched state can be affixed to a deflated inflatable cylinder 310 such that the protective surface 360 stretches with the inflating inflatable cylinder 310 without separating from the inflatable cylinder 310 or deforming the exterior portion 312 of the inflatable cylinder 310 as it inflates. Alternatively, the protective surface 360 in a stretched state can be affixed to an inflated inflatable cylinder 310 such that the protective surface 360 contracts with the inflatable cylinder 310 as it deflates and does not separate from the inflatable cylinder 310 or deform the exterior portion 312 of the inflatable cylinder 310.

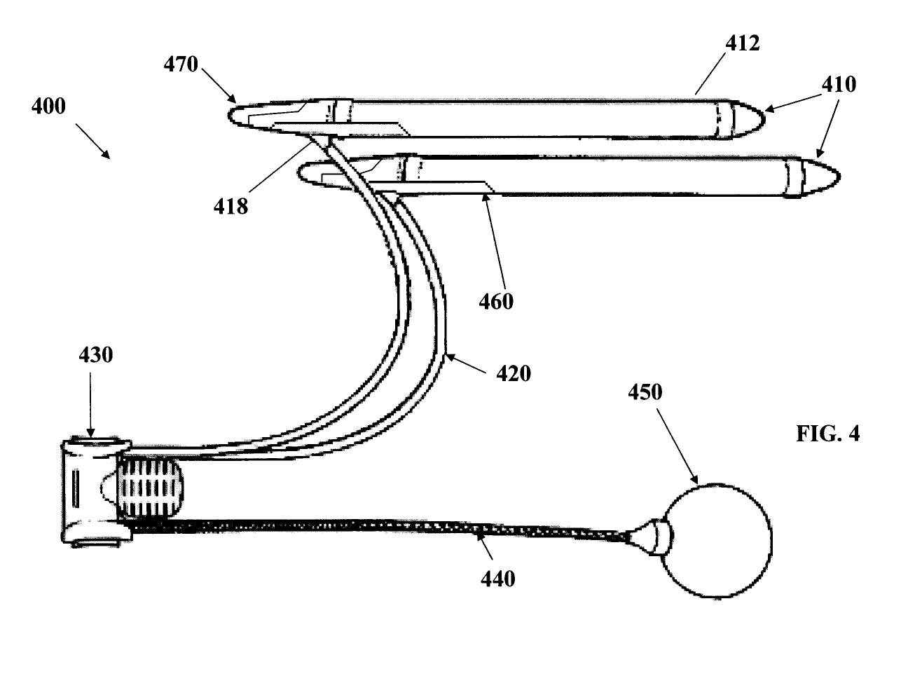

As illustrated in FIG. 4, another representative embodiment of an implantable penile prosthesis 400 can further comprises a reservoir 450 linked to a pump 430 by kink resistant tubing 440. The implantable penile prosthesis 400 further comprises a first protective surface 460 disposed on the inflatable cylinders 410 surrounding around the junction 418 of the inflatable cylinder 410 and the kink resistant tubing 420 linking the inflatable cylinder 410 to the pump 430. The first protective surface 460 protects the inflatable cylinders 410 from damage caused by repeated contact with the kink resistant tubing 420. The inflatable cylinders 410 further comprise a second protective surface 470 arranged on an exterior portion 412 of the inflatable cylinder 410 to prevent the kink resistant tubing 440 joining the reservoir 450 to the pump 430 from damaging the inflatable cylinder 410. First and second protective surfaces 460, 470 can comprise similar materials as the protective surface 360 and can be similarly affixed to each inflatable cylinder 410.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative embodiments.

1. An implantable penile prosthesis comprising:
an inflatable cylinder having an exterior portion;
a pump adapted to inflate the inflatable cylinder;
a first length of kink resistant tubing providing fluid communication between the inflatable cylinder and the pump, wherein at least a portion of the first kink resistant tubing is capable of contacting the exterior portion of the inflatable cylinder; and
a first protective surface disposed on the exterior surface of the inflatable cylinder, the first protective surface comprising a low friction material positioned to protect the inflatable cylinder from damage due to repeated contact between the first kink resistant tubing and the exterior portion of the inflatable cylinder.

2. The implantable penile prosthesis of claim 1, further comprising:
a reservoir for providing an inflation fluid to the pump for inflating the inflatable cylinder;
a second length of kink resistant tubing providing fluid communication between the reservoir and the pump, wherein at least a portion of the second kink resistant tubing is capable of contacting the exterior portion of the inflatable cylinder; and
a second protective surface disposed on the exterior portion of the inflatable cylinder, the second protective surface comprising the low friction material positioned to protect the inflatable cylinder from damage due to repeated contact between the second kink resistant tubing and the exterior portion of the inflatable cylinder.

3. The implantable penile prosthesis of claim 1, wherein the first protective surface is shorter in width than a predetermined zone of inhibition for an antibiotic coating.

4. The implantable penile prosthesis of claim 1, wherein the protective surface stretches and contracts as the inflatable cylinder changes in size, such that the protective surface remains affixed to the inflatable cylinder.

5. The implantable penile prosthesis of claim 1, wherein the protective surface covers the entire exterior surface of the inflatable cylinder.

6. The implantable penile prosthesis of claim 1, wherein the low friction material comprises polytetrafluorethylene, poly-(p-xylylene) and combinations thereof.

7. The implantable penile prosthesis of claim 1, wherein the protective surface has a maximum width of 2 cm.

8. The implantable penile prosthesis of claim 7, wherein the protective surface has a width of between 1 cm to 2 cm.

9. A method of protecting an inflatable cylinder of an implantable penile prosthesis from damage resulting from repeated contact of kink resistant tubing with the inflatable cylinder, comprising:
providing a protective surface comprising a low friction material; and
affixing the protective surface to an exterior portion of an inflatable cylinder, the protective surface positioned to bear repeated contact by a kink resistant tubing.

10. The method of claim 9, wherein the implantable penile prosthesis comprises an antibiotic coating, the method further comprising:
sizing the protective surface such that the protective surface is smaller in width than a predetermined zone of inhibition for the antibiotic coating.

11. The method of claim 9, further comprising:
sizing the protective surface to cover an exterior portion of the inflatable cylinder likely to contact the kink resistant tubing.

12. The method of claim 9, further comprising:
affixing a second protective surface on the exterior portion of the inflatable cylinder, the second protective surface comprising the low friction material, wherein the second protective surface protects the inflatable cylinder from damage due to repeated contact between a second kink resistant tubing and the exterior portion of the inflatable cylinder.
